(19) United States
(12) Patent Application Publication
(10) Pub. No.: US 2025/0052693 A1
(43) Pub. Date: Feb. 13, 2025

(54) METHOD AND DEVICE FOR INSPECTING EGGS CONTACTLESSLY

(71) Applicant: EGG-CHICK AUTOMATED TECHNOLOGIES, LANDIVISIAU (FR)

(72) Inventors: Frédéric BERIER, LANDIVISIAU (FR); Laura TRUBUIL, LANDIVISIAU (FR); Hugo REVOIS, LANDIVISIAU (FR)

(21) Appl. No.: 18/693,876

(22) PCT Filed: Sep. 19, 2022
(86) PCT No.: PCT/FR2022/051758

§ 371 Date: Oct. 9, 2024

(30) Foreign Application Priority Data
Sep. 22, 2021 (FR) FR2109994

(51) Int. Cl. G01N 22/00; G01N 33/08

(52) U.S. Cl. CPC G01N 22/00; G01N 33/08

(57) ABSTRACT

The present invention relates to a method and device for automatically inspecting one or more eggs contactlessly. According to the invention, the following steps are carried out: a) a millimeter-wave radio-frequency signal is transmitted by a transmitter towards an egg, b) a millimeter-wave radio-frequency signal reflected by said egg is detected by means of a sensor (14), said sensor (14) being placed at distance from said egg, c) the intensity of the reflected millimeter-wave radio-frequency signal is analyzed as a function of the distance traveled by the reflected signal and the radar echo thus obtained is compared with one or more reference radar echoes each representative of one egg state, so as to deduce the egg's current state therefrom.

### TECHNICAL FIELD

The present invention relates to a method for automatically contactlessly inspecting eggs placed in containers on a treatment line.

It also relates to an device for implementing such an inspection method.

### PRIOR ART

It is known in the field of poultry farming, in particular in chick production, to use the optical properties of the eggs to differentiate between them and bypass during processing the eggs identified as being unlikely to hatch and produce a chick.

The latter are essentially infertile eggs or fertilized eggs but whose egg embryo is dead or else malformed.

This differentiation is necessary to minimize the losses of vaccines during in ovo treatment, that is, during injection of the egg by vaccine needle through the shell in order to promote its development after hatching and to prevent the appearance of diseases. It is also necessary in order to avoid the explosion of rotten eggs, which might contaminate viable surrounding eggs in the container and the injection material that can be used to inject these viable eggs, which would also risk contaminating the latter.

Note that the explosion of rotten eggs is also likely to cause a mess on the optical protection screens used in egg differentiation, the method associated therewith commonly being called “candling”.

Now, the mess can be detrimental to the quality of detecting the state of certain eggs when they remain light, or even prevent such detection if the mess is greater. The machine used to perform the candling, referred to as a candling machine, must therefore be stopped to ensure its cleaning.

It is further observed that this candling method is sensitive to the outside environment, light sources such as sunlight or halogen lighting being likely to disrupt the measurements obtained during the step of candling the eggs contained in a basket.

This method is also sensitive to the state of messiness of the measured eggs.

Additionally, such a candling method allows only a low treatment throughput.

Moreover, methods are known for identifying eggs arranged upside down in a batch of eggs.

Such a detection of eggs in the upside down position is necessary to ensure that the needle of the injector remains oriented towards the air chamber of the corresponding egg during an in-ovo injection of a vaccine or nutrients, failing which the embryo may be damaged or even killed.

For example, a method of the prior art for identifying eggs arranged upside down in a batch of eggs consists in heating this batch of eggs with a source of radiation.

The eggs are then thermally imaged while they are no longer exposed to the radiation source, and the thermal images thus captured are analyzed to detect the presence of a hot zone and to identify the eggs arranged upside down, or in the inverted position.

Indeed, since the air chamber of an egg acts as an insulator, when an egg placed upside down is subjected to thermal radiation, the temperature of its shell around this air chamber increases. On the contrary, when the egg is right-side up, the heat generated by the exposure of its shell to thermal radiation is dissipated in the liquids present inside the egg and the shell of the latter then appears “cold”.

However, such a method requires a preliminary egg heating step that is time-consuming and reduces the treatment throughputs likely to be reached on the treatment line, these throughputs typically not exceeding 70,000 eggs/h.

In addition, this prior heating step is typically carried out with halogen-type flash lamps that have a power greater than 3,000 W, or even 5,000 W and more, to provide a detectable temperature rise of the shell of the egg placed upside down, without significantly heating the rest of this egg. Indeed, the increase in temperature of the other parts of the egg, that is, yolk, amniotic fluid and embryo, must remain negligible.

Now, the energy consumption of these light sources is very significant and therefore expensive.

There is therefore a pressing requirement for a method for inspecting eggs placed in container divots, the original design of which makes it possible to overcome the disadvantages of the prior art described above.

### SUBJECT MATTER OF THE INVENTION

The present invention aims to overcome the disadvantages of the prior art by proposing a method and a device for contactlessly inspecting eggs placed in containers, simple in design and in operating mode, cost-effective and not sensitive to the outside environment or to the state of cleanliness of the eggs to be measured.

Another object of the present invention is such a contactless inspection method and device allowing extremely fast rates, and by way of illustration, of more than 90,000 eggs per hour.

Yet another subject matter of the present invention is such a contactless inspection method and device that is safer for the embryos of the eggs, and as a result, promoting the hatching of these eggs to ensure greater yields.

### DISCLOSURE OF THE INVENTION

To this end, the invention relates to a method for the contactless inspection of an egg. According to the invention, the following steps are carried out:
a) a millimeter-wave radio-frequency signal is transmitted by a transmitter towards an egg,
b) a millimeter-wave radio-frequency signal reflected by said egg is detected by means of a sensor, said sensor being placed at a distance from said egg,
c) the intensity of the reflected millimeter-wave radio-frequency signal is analyzed based on the distance traveled by the reflected signal and the radar echo thus obtained is compared with one or more reference radar echoes each representative of one egg state, so as to deduce the egg's current state therefrom.

The original design of this method for contactlessly inspecting eggs in a batch or container, or else a tray, which implements radar waves in the frequency range of millimeter waves allows extremely fast rates, typically greater than 90,000 eggs per hour and more.

This inspection method is also not sensitive to the outside environment, for example to parasitic lights, to parasitic heat sources or to environmental temperature variations.

This inspection method allows, regardless of the cleanliness state of the egg to be inspected, the determination of:
the positioning right-side up or upside-down positioning of the egg, which is reliable and fast, or
the viable or non-viable state of the egg, this determination requiring the egg to be incubated since it is based on the detection of an optional movement of the embryo. This measurement is based on the measurement of low variation in the phase of the signal reflected by the embryo inside the egg. The measurement of the phase fluctuations is based on analyzing radar echoes integrated into the implemented sensor.

According to one embodiment of this method for contactlessly inspecting an egg, the transmitter and the sensor are positioned at the same distance, or substantially at the same distance, from the egg by being arranged coaxially.

Alternatively, it is of course possible that the transmitter and the sensor are not placed at the same distance from the target.

According to another embodiment of this method for contactlessly inspecting an egg, the egg being in a fixed position determining a first end and a second end of said egg, the egg having an air chamber that can be placed either at said first end or at said second end, one of these ends determining an upside-down or inverted arrangement of the egg when the air chamber is placed at this end, the position of the egg is detected to identify a possible upside-down arrangement of this egg.

Alternatively, a viable, not viable, i.e. non-fertilized or dead, uncertain state of the egg thus analyzed or an absence of egg is determined.

Advantageously, it is then sought to determine, for each fertilized egg comprising an embryo, whether that embryo is living or dead, or whether it is malformed or too small for its age.

Such a state then being detected, the corresponding egg will advantageously be ignored in the rest of the treatment of the container, in particular in a step for selective injection of the eggs.

If the divot of the tray, or container, is empty, this divot is also bypassed in the remainder of the treatment of the container.

According to yet another embodiment of this method for contactlessly inspecting an egg, the egg being placed in a divot of a tray transported by a conveyor, said transmitter is arranged so that said conveyor moves said egg under, or above, said transmitter capable of transmitting a millimeter-wave radio-frequency signal, said transmitter being centered or substantially centered on said divot receiving the egg to be analyzed.

Of course, and purely by way of illustration, a first transmitter can be arranged to come above an egg, while a second transmitter is arranged to come below the same egg in order to acquire two radar echoes that will be analyzed in order to have a more precise measurement.

The millimeter waves correspond to the frequency range between 30 GHz and 300 GHz.

According to yet another embodiment of this method for contactlessly inspecting an egg, in step a), a millimeter-wave radio-frequency signal is transmitted in the frequency range between 30 and 300 GHz, and even better still between 150 and 300 GHz, and even more preferentially between 200 and 300 GHz.

Implementing high frequencies improves the accuracy of the measurements.

Alternatively, a frequency of between 50 and 70 GHz could be used, and better still a frequency of, or around, 60 GHz.

These frequencies advantageously undergo a strong path attenuation that is proportional to the frequency. Furthermore, and advantageously, a frequency of 60 GHz (0.5 mm wavelength) is used very little and is absent from the environmental electromagnetic diagram. No external pollution is likely to disrupt the radar measurements produced.

According to yet another embodiment of this method for contactlessly inspecting an egg, in step a), a focused beam of millimeter waves is transmitted onto said egg.

Preferably, a focusing lens and even better a convex lens is used. This convex lens can advantageously be obtained by three-dimensional printing.

Advantageously, this focusing lens is positioned relative to the transmitter so that the divergence of the millimeter-wave beam transmitted by this transmitter is less than 10°, and even better still strictly less than 8°, and is preferably less than or equal to 6°.

According to yet another embodiment of this method for contactlessly inspecting an egg, the detection of the millimeter-wave radio-frequency signal reflected by the egg is carried out in the absence of a transmission of a millimeter-wave radio-frequency signal towards said egg.

This avoids mixing the incident radio-frequency signal and the radio-frequency signal reflected by the egg.

In an alternative embodiment, a sensor can be implemented that operates in continuous transmission, with an interferometric detection means for analyzing the delay and phase shift of the radar echo.

According to yet another embodiment of this method for contactlessly inspecting an egg, an additional step of marking the non-viable and upside-down eggs and/or an additional step of reorienting the eggs arranged upside down is carried out.

According to yet another embodiment of this method for contactlessly inspecting an egg, in step c) the data related to a given container are processed, the information obtained by this processing being stored in and/or sent to a remote egg treatment station such as a device for delivering in-ovo injection of eggs, so that this remote treatment station receiving said container of eggs to be treated has information necessary for its treatment.

This thus ensures continuity in the processing of each container, the next treatment station on the high-throughput treatment line having already received, from the contactless inspection device, the information relating to the container to be treated before it is taken in.

According to yet another embodiment of this inspection method, the acquisition of radar echoes for a container and the processing of the data related to radar echoes are carried out in parallel so that the processing of the data related to radar echoes obtained for a first container is carried out while radar echoes are acquired for the next immediately adjacent container on said conveyor

According to yet another embodiment of this method for contactlessly inspecting an egg, said trays are transported at a constant speed V by a straight conveyor.

Insofar as each container moves in translation, in particular at a constant speed on a straight conveyor, which, at a high throughput, has the advantage of avoiding jerking, the eggs remain stable in their respective divots of the container and consequently have optimal positioning for their subsequent injection.

By way of example, this is an endless belt conveyor.

According to yet another embodiment of this method for contactlessly inspecting an egg, said trays are transported at a speed strictly greater than or equal to 1 m/min, and even better still 10 m/min, and even more preferentially of about 15 m/min, being spaced apart by a safety distance at least equal to d=100 mm to ensure a high treatment throughput.

According to yet another embodiment of this method for contactlessly inspecting an egg, during the measurement of the eggs contained in said container, it is determined by means of a position sensor, the length of said container being measured, the thus measured length of said container is compared with its real length and the absence or existence of an inadvertent movement of said container during step a) and/or step b) of the contactless inspection method.

Advantageously, the original design of this step allows simple and inexpensive detection of an inadvertent movement of a container transported by a conveyor, this movement resulting in a loss of its exact position on the conveyor during a measurement of the eggs thereby transported.

Preferentially, this position sensor being arranged to detect the front and rear ends of a container moving on said conveyor, the time interval separating the detection by this sensor of said ends is measured, and a measured length of the container is calculated by the product of this time interval multiplied by the driving speed of that container along said treatment line.

Advantageously, this position sensor is arranged to detect those ends of a container when they pass right next to that sensor during the transport of the container along the treatment line.

Alternatively, this position sensor being arranged to detect the front and rear ends of a container moving on said conveyor, the number of encoder points elapsed between the detection of these front and rear ends by said sensor is determined, and this number of encoder points is converted into the measured length of said container.

As the distance traveled by the belt of the conveyor during an encoder run is known, the number of encoder points thus determined can easily be translated or converted into a distance.

Recall that the number of encoder points per encoder is linked to the resolution of this encoder.

The encoder advantageously emits an electrical signal giving the number of encoder points produced between the detection of the two front and rear ends. Advantageously, this measurement of the length of the container is thus independent of the drive speed of the conveyor.

During the comparison step, it is also possible to take into account a previously determined tolerance range over the measured length of the container.

According to yet another embodiment of this method for contactlessly inspecting an egg, in step c) and before comparison, a processing step is carried out to remove any interference signals in the radar echo obtained. Advantageously, only the useful signal from the egg thus inspected is retained. These interference signals can come from the container for which the high points/the thick edges can emerge from the radar echo.

Different processing methods can be envisaged to eliminate these interference signals, such as
reducing the useful data area of the radar echo for the classification: for example, by keeping only the echoes of useful depths (top half of the egg), or by removing the samples from the edges of the egg.
carrying out a measurement of an “empty container, or tray” and taking the signal thus obtained as reference to “subtract” to the signals acquired when the eggs are scanned.

The present invention also relates to a device for automatically contactlessly inspecting eggs, such as poultry eggs, comprising, for each egg, a radar module configured to transmit millimeter waves towards said egg and to detect millimeter waves that are reflected by said egg, said radar module transmitting output signals from said reflected millimeter waves thus detected, said measurement device comprising a processing unit for analyzing said output signals and deducing a state of the corresponding egg therefrom.

Such an apparatus advantageously allows contactless inspection of eggs arranged in a container, or basket, while being safer for the embryos of the eggs.

This apparatus is particularly suitable for the high-throughput treatment of objects on an industrial automatic line for treating objects having fragile content.

According to one embodiment of this device for contactlessly inspecting eggs, each radar module comprises a lens for focusing the millimeter wave beam over the corresponding egg, said focusing lens preferably being a convex lens.

According to another embodiment of this device for contactlessly inspecting eggs, said radar module is configured to send a millimeter-wave radio-frequency signal towards the egg in the frequency range between 30 and 300 GHz, and even better still between 150 and 300 GHz, and even more preferentially between 200 and 300 GHz.

According to still another embodiment of this device for contactlessly inspecting eggs, each radar module is configured to transmit a millimeter wave beam having a power of less than 0.15 mW/cm², and even better still less than or equal to 0.1 mW/cm² to avoid any risk for the development of the embryo.

According to yet another embodiment of this device for contactlessly inspecting eggs, said radar module comprises a first antenna for transmitting a millimeter wave beam towards said egg and a second antenna for receiving the millimeter waves reflected by said egg, said first and second antennas being carried by a same support while being coaxial.

According to yet another embodiment of this device for contactlessly inspecting eggs, it comprises a straight conveyor for moving trays comprising divots arranged in rows and columns, each row comprising n divots, said conveyor defining a conveying axis, said device comprising n radar modules aligned along a same measurement axis that is perpendicular, or substantially perpendicular, to said conveying axis, said radar modules being spaced apart from one another by an equal or substantially equal distance to come above and/or below a single one of the divots of said row when the latter is placed below and or above, respectively, said radar modules.

Advantageously, this inspection device comprises a control unit inspecting the transport speed of the trays by the conveyor, which is configured to define a constant transport speed of these trays along a conveying path. More generally, the movement of the trays on the straight conveyor is carried out without jerking.

The constant-speed movement ensures in particular the stability of the eggs in their divots and as a result, an optimal orientation of these eggs for their subsequent treatment on other stations of a high-speed treatment line.

Preferably, the device also comprises at least one position sensor placed upstream of said radar modules on said conveyor and connected to a central unit so as to launch a data acquisition cycle for an egg tray whose downstream end is detected in a first position defined by said position sensor, said central unit being configured to trigger said millimeter-wave transmissions on each passage of a row of the egg tray during acquisition.

Advantageously, each position sensor is a photoelectric cell that is, for example, placed on the conveyor belt side of the conveyor.

According to yet another embodiment of this device for contactlessly inspecting eggs, each radar module is arranged to be centered, or substantially centered, on the axis of symmetry of the corresponding divot when this divot of the tray being acquired passes under, and/or above, respectively, a radar module.

The device for contactlessly inspecting eggs can thus comprise, for each corresponding divot, a first radar module intended to be placed above the divot and a second radar module intended to be positioned simultaneously below this divot when the tray moves between these radar modules.

According to yet another embodiment of this device for contactlessly inspecting eggs, it comprises a communication module for sending data or information obtained by processing radar echoes of the eggs of a given tray to a remote station such as a device for in-ovo injection of the eggs of this tray.

According to yet another embodiment of this device for contactlessly inspecting eggs, it comprises means for marking non-viable eggs and those arranged upside down. It may also comprise a device for reorienting eggs arranged upside down so as to once again place them right-side up in their divot.

### BRIEF DESCRIPTION OF THE DRAWINGS

Other advantages, aims and particular features of the present invention will become apparent from the following description, made, for explanatory purposes and in no way limiting, with reference to the appended drawings, in which:

FIG. 1 is a partial schematic representation of a device for contactlessly inspecting eggs arranged in baskets transported by a conveyor according to a particular embodiment of the present invention, the egg here being positioned right-side up in its divot;

FIG. 2 shows the detection of an egg positioned upside down, or inverted, in its divot with the device for contactlessly inspecting eggs shown in FIG. 1;

FIG. 3 is a schematic and partial view of a tray traveling under a radar module of the device for contactlessly inspecting eggs of FIG. 1, the triggering of the measurement being carried out when the front edge of the divot receiving the egg to be measured passes right next to the radar module;

FIG. 4 is a schematic and partial view of the tray of FIG. 3 at a later instant in its transport on the conveyor, the stopping of the measurement being carried out when the rear edge of the divot receiving the egg to be measured passes right next to the radar module;

FIG. 5 is a time diagram obtained with the device for contactlessly inspecting eggs shown in FIG. 1 for an egg positioned right-side up in its divot;

FIG. 6 is a time diagram obtained with the device for contactlessly inspecting eggs shown in FIG. 1 for an egg positioned upside down in its divot;

FIG. 7 is a time diagram obtained with the device for contactlessly inspecting eggs shown in FIG. 1 with no egg in the divot;

FIG. 8 shows a set of measurements carried out with the device for contactlessly inspecting eggs shown in FIG. 1, for fifteen eggs on the fourteenth day, the eggs being positioned right-side up in their respective divots;

FIG. 9 shows a set of measurements carried out with the device for contactlessly inspecting eggs shown in FIG. 1, for fifteen eggs on the fourteenth day, the eggs being positioned upside down, or being inverted, in their respective divots;

### DESCRIPTION OF EMBODIMENTS

The drawings and the following description essentially contain elements of a certain nature. They may therefore not only serve to better understand the present invention, but also contribute to its definition, where appropriate.

First, it should be noted that the figures are not to scale.

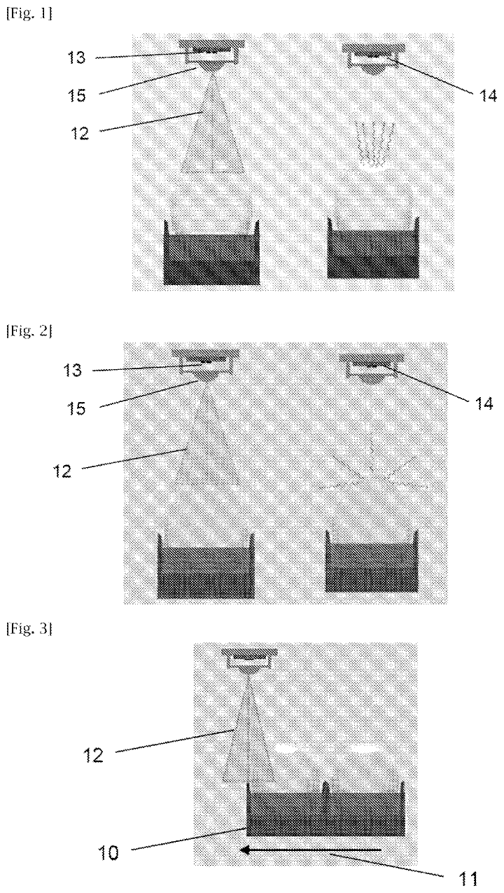

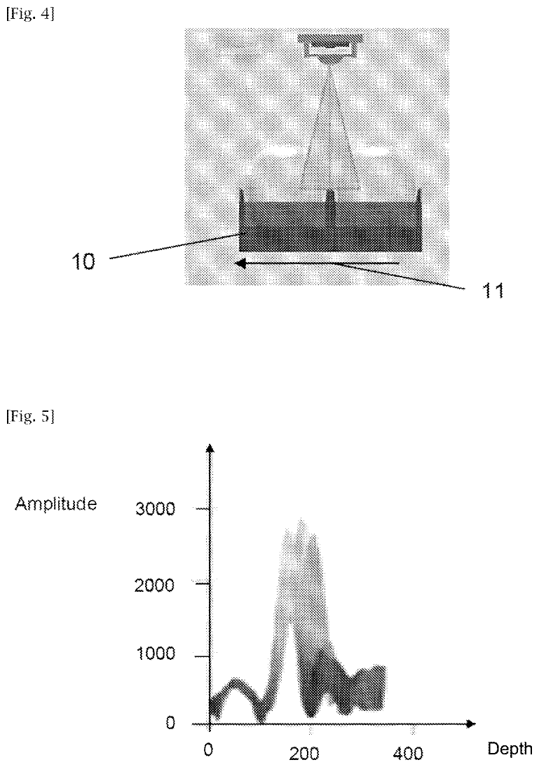

FIGS. 1 to 4 schematically show a device for contactlessly inspecting eggs arranged in moving baskets 10 on a straight conveyor 11 in a particular embodiment of the present invention.

This straight conveyor 11, which here is of the endless belt type, comprises a control unit (not shown) controlling the transport speed of the baskets 10.

Advantageously, these baskets 10 are moving at constant speed to avoid causing jerking that is likely to cause the eggs to move around and/or cause impacts to the embryos of these eggs.

These moving baskets 10, which have a general “rectangular” shape, comprise a plurality of divots, or cells, in each of which an egg is normally received. These divots are distributed in rows and columns, each row here comprising ten (10) divots.

These baskets 10 are advantageously made of a material that is transparent to millimeter waves, such as a plastic material.

Preferably, the cells of these baskets 10 have a flared shape with their open upper end as wide as possible so that the edges of this opening do not encounter the millimeter-wave beam 12 sent towards the corresponding egg.

These eggs are preferably oriented in their divot for their injection in-ovo, that is that their narrowest end is arranged downward so that the air chamber is arranged upward. This position of the egg is considered to be “right-side up”. The risks of the injection needle damaging the embryo of the egg are thus reduced. The egg is preferably oriented vertically in its divot. However, sometimes eggs are positioned incorrectly, or inverted, in their respective divots. If the egg is inverted (that is, with its air chamber down), the egg is said to be “upside down”.

The device disclosed in this document makes it possible to inspect the orientation of the eggs very simply and quickly.

This contactless inspection device comprises ten radar modules, one per divot of a row of the basket 10, these radar modules being aligned by being spaced apart from one another so that a single radar module is located above one divot at a time. Preferably, it will be ensured that each radar module of the corresponding row is centered or substantially centered over its divot in order to perform a measurement. Note that it is not necessary for the air chamber of the egg to be centered relative to the radar module. However, the signal of the millimeter waves reflected by the egg is maximal in the centered configuration of the air chamber.

This radar module comprises a first antenna 13 for emitting a millimeter-wave beam 12, at a frequency of 60 GHz, towards the corresponding egg. It also comprises a second antenna 14 for receiving the millimeter waves reflected by this egg.

These first and second antennas 13, 14 are carried by the same support while being arranged coaxially. This radar module also comprises a convex lens 15 for focusing the millimeter wave beam 12 over the corresponding egg.

Advantageously, the divergence of the millimeter-wave beam 12 sent towards said egg is of the order of 6° to encounter only the egg.

The form of the liquid/air interface, by reflecting the millimeter waves, will make it possible to identify the right-side up or upside down positioning of the corresponding egg in its divot. The shell is considered to be transparent at these frequencies between 30 GHz and 300 GHz.

The wave is reflected in all cases by the surface of the amniotic fluid, or the allantoic fluid depending on the developmental stage. The incident millimeter-wave beam 12 would be reflected without deformation if the liquid surface was flat. It would then be non-dispersed. The intensity measured in return would only be dependent on the distance between the egg and the second detection antenna 14 and on the cross section of the egg.

Thus, and as shown in FIG. 1, when the egg is in the right-side up position in its cell, it shows its rounded part and the surface of the liquid is concave. The intensity of the signal related to the detection of the millimeter-wave beam reflected by the egg is high.

When the surface of the amniotic fluid, or the allantoic fluid, is convex (FIG. 2), the incident millimeter-wave beam 12 is dispersed and as a result the intensity of the signal related to the detection of the reflected millimeter-wave beam, which returns to the second antenna 14, is reduced. The egg is arranged upside down, or is inverted.

The baskets 10 are supplied on the straight conveyor 11 at a regular minimum intervals by being aligned in a row. They thus have minimum spacing between them.

FIGS. 5 to 7 show examples of time diagrams produced by the device for contactlessly inspecting eggs shown in FIGS. 1 to 4.

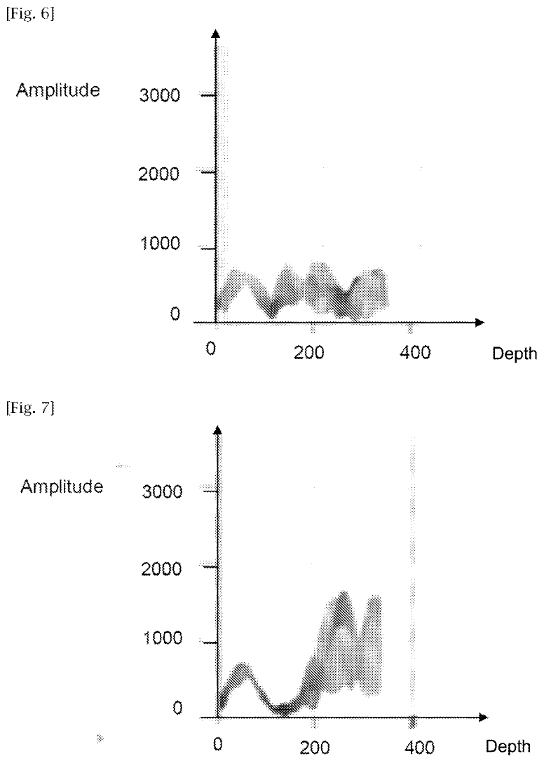

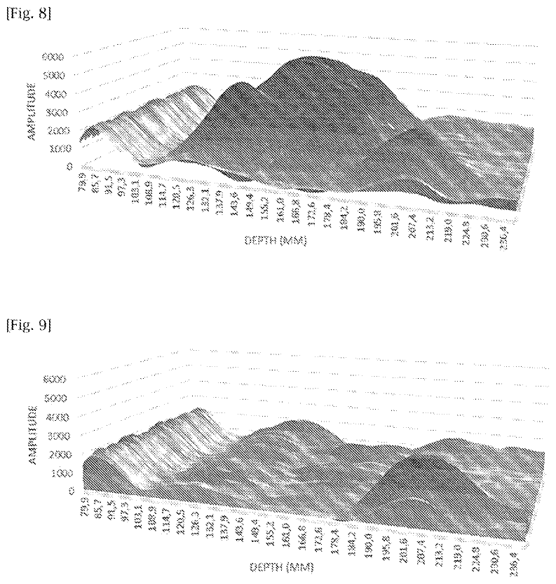

Owing to the radar signature of each egg, it is possible to very reliably determine the orientation of this egg in its divot (FIGS. 5 and 6), or even the absence of egg in the corresponding divot (FIG. 7).

This determination is carried out comparing the measured radar echo for the egg to be inspected with reference radar echoes that have been previously recorded in a data library stored on a storage unit.

For example, the time diagram shown in FIG. 5 corresponds to an egg that is correctly positioned in its divot, while the time diagram shown in FIG. 6 corresponds to an egg that is positioned upside down, or inverted, in its divot.

It has been observed that the identification of the upside down or right-side up position of the egg in its divot by means of the contactless inspection device disclosed above is very easy.

Identification rates of the order of 100% are achieved, showing the benefit of the present invention.

1. A method for contactlessly inspecting an egg, the method comprising:
a) a millimeter-wave radio-frequency signal is transmitted by a transmitter towards an egg,
b) a millimeter-wave radio-frequency signal reflected by said egg is detected by means of a sensor, said sensor being placed at a distance from said egg, and
c) the intensity of the reflected millimeter-wave radio-frequency signal is analyzed based on the distance traveled by the reflected signal and the radar echo thus obtained is compared with one or more reference radar echoes each representative of one egg state, so as to deduce the egg's current state therefrom.

2. The method according to claim 1, wherein the transmitter and the sensor are positioned at the same distance, or substantially at the same distance, from the egg by being arranged coaxially.

3. The method according to claim 1, wherein, with the egg being in a fixed position, determining a first end and a second end of said egg, the egg having an air chamber that can be placed either at said first end or at said second end, one of these ends determining an upside-down arrangement of the egg when the air chamber is placed at this end, the position of the egg is detected to identify a possible upside-down arrangement of this egg.

4. The method according to claim 3, wherein a viable, not viable, i.e. non-fertilized or dead, uncertain state of the egg thus analyzed or an absence of egg is determined.

5. The method according to claim 1, wherein the egg being placed in a divot of a tray transported by a conveyor, said transmitter is arranged so that said conveyor moves said egg under, or above, said transmitter capable of transmitting a millimeter-wave radio-frequency signal, said transmitter being centered or substantially centered on said divot receiving the egg to be analyzed.

6. The method according to claim 1, wherein in step a), a millimeter-wave radio-frequency signal is transmitted in the frequency range between 30 and 300 GHz.

7. The method according to claim 4, further comprising marking the non-viable and upside-down eggs and/or reorienting the eggs arranged upside down.

8. A device for automatically contactlessly inspecting eggs, comprising, for each egg, a radar module configured to transmit millimeter waves towards said egg and to detect millimeter waves that are reflected by said egg, said radar module transmitting output signals from said reflected millimeter waves thus detected, said measurement device comprising a processing unit for analyzing said output signals and deducing a state of the corresponding egg therefrom.

9. The device for automatically contactlessly inspecting eggs according to claim 8, wherein each radar module comprises a lens for focusing the millimeter-wave beam onto the corresponding egg, said focusing lens preferably being a convex lens.

10. The device for automatically contactlessly inspecting eggs according to claim 8, wherein said radar module is configured to send a millimeter-wave radio-frequency signal towards the egg in the frequency range between 30 and 300 GHz.

11. The device for automatically contactlessly inspecting eggs according to claim 8, wherein each radar module is configured to transmit a millimeter wave beam having a power of less than 0.15 mW/cm² to avoid any risk for the development of the embryo.

12. The device for automatically contactlessly inspecting eggs according to claim 8, wherein said radar module comprises a first antenna for transmitting a millimeter wave beam towards said egg and a second antenna for receiving the millimeter waves reflected by said egg, said first and second antennas being carried by a same support while being coaxial.

13. The device for automatically contactlessly inspecting eggs according to claim 8, further comprising a straight conveyor for moving trays comprising divots arranged in rows and columns, each row comprising n divots, said conveyor defining a conveying axis, said device comprising n radar modules aligned along a same measurement axis that is perpendicular, or substantially perpendicular, to said conveying axis, said radar modules being spaced apart from one another by an equal or substantially equal distance to come above and/or below a single one of the divots of said row when the latter is placed below and or above, respectively, said radar modules.

14. The device for automatically contactlessly inspecting eggs according to claim 13, further comprising a position sensor placed upstream of said radar modules on said conveyor and connected to a central unit so as to launch a data acquisition cycle for an egg tray whose downstream end is detected in a first position defined by said position sensor, said central unit being configured to trigger said millimeter-wave transmissions on each passage of a row of the egg tray during acquisition.

15. The device for automatically contactlessly inspecting eggs according to claim 14, wherein each radar module is arranged to be centered, or substantially centered, on the axis of symmetry of the corresponding divot when this divot of the tray being acquired passes under, and/or above, respectively, a radar module.
